(19) United States
(12) Patent Application Publication
(10) Pub. No.: US 2023/0301569 A1
(43) Pub. Date: Sep. 28, 2023

(54) A METHOD AND DEVICE FOR PROVIDING AN AUTOMATED PREDICTION OF A CHANGE OF A STATE OF FATIGUE OF A SUBJECT CARRYING OUT A VISUAL TASK

(71) Applicant: Essilor International, Charenton-Le-Pont (FR)

(72) Inventors: Yi GAO, Singapore (SG); Daniel SPIEGEL, Singapore (SG); Bjorn DROBE, Singapore (SG)

(73) Assignee: Essilor International, Charenton-Le-Pont (FR)

(21) Appl. No.: 18/042,253

(22) PCT Filed: Aug. 30, 2021
(86) PCT No.: PCT/EP2021/073823

§ 371 Date: Feb. 20, 2023

(30) Foreign Application Priority Data
Sep. 14, 2020 (EP) 20306024.9

(51) Int. Cl. A61B 5/16; G16H 50/30; G16H 10/20

(52) U.S. Cl. CPC A61B 5/163; G16H 50/30; G16H 10/20; A61M 2021/0044

(57) ABSTRACT

This computer-implemented method for providing an automated prediction of a change of a state of fatigue of a subject carrying out a visual task involving any kind of visual content comprises: providing a plurality of input data, relating to the subject, to a fatigue state change predictive model, wherein the plurality of input data comprises at least one subjective measurement relating to the subject and/or at least one objective measurement relating to the subject and/or at least one other subject-related datum; obtaining, by a processor implementing the model, a value representing a level of change of the state of fatigue of the subject.

### FIELD OF THE DISCLOSURE

The present disclosure relates to a method and device for providing an automated prediction of a change of a state of fatigue of a subject carrying out a visual task.

The present disclosure also relates to an associated optometric machine, computer program product and mobile terminal, as well as to a corresponding method for providing a subject with a personalized prescription of an anti-fatigue optical article.

### BACKGROUND OF THE DISCLOSURE

Visual fatigue and the terms “computer vision syndrome” and “digital eye strain” are often used interchangeably to describe the complex eye and vision-related problems that result from prolonged near-vision work on digital devices such as computers, tablets, cell phones and e-readers.

More generally, other types of fatigue may also result from a prolonged visual task:
cognitive fatigue, i.e. brain tiredness, which is for example expressed by a change in attention level or in the reading speed;
general fatigue, for example feeling sleepy or generally tired;
muscular fatigue, for example neck pain, due to holding the same posture for a long time;
driver fatigue;
etc.

Due to the modern lifestyle, the usage of digital devices as well as the prevalence of such types of fatigue has been dramatically increasing to a very high degree in the last two decades.

Regarding visual fatigue, two official definitions are given below:
the World Health Organization defines (ICD-10, H53.1) visual fatigue as subjective visual disturbance, manifested by a high degree of visual discomfort typically occurring after prolonged visual activity, and characterized by fatigue, pain around the eyes, blurred vision and headache; and
the American Optometric Association defines visual fatigue by the above-mentioned computer vision syndrome, which describes a group of eye and vision-related problems that result from prolonged computer, tablet, e-reader and cell phone use.

The symptoms, assessment techniques and management strategies of visual fatigue have attracted much attention in the research and medical communities. The symptoms may be caused by poor lighting, glare on a digital screen, improper viewing distances, long work hours, alert levels, light spectrum, reduced contrast, small font, uncorrected vision problems, etc.

The methods for visual fatigue assessment are classified as subjective or objective.

For subjective assessment, questionnaires may be used. Most of commonly used questionnaires are symptom-based questionnaires, such as the Visual Discomfort Scale (Conlon, Lovegrove, Chekaluk & Pattison, 1999, available at https://www.tandfonline.com/doi/abs/10.1080/135062899394885), a 10-item visual fatigue questionnaire (Hayes, Sheedy, Stelmack & Heaney, 2007, available at https://www.researchgate.net/publication/6140024_Computer_Use_Symptoms_an d_Quality_of_Life) and the Convergence Insufficiency Symptom Survey (CISS) (available at https://wowvision.net/wp-content/uploads/2014/08/CI-Screening-and-symptom-survey.pdf).

For objective assessment, accommodation micro-fluctuation may be monitored, as it may be found to increase after a prolonged visual task. Some studies assessed accommodative response or accommodative lag after inducing visual discomfort, but found no consistent change in those parameters after a prolonged visual task. Vergence dynamics, associated phoria and near point of convergence were found to change after near computer work. Blinking pattern was found to show reduced blink rate, increased blink duration and increased incomplete blinking. Pupil diameter change, increased pupil micro-fluctuation, increased pupillary reflexes were also identified as a potential indicator of visual fatigue. Critical Flicker Fusion Frequency (CFFF) was found to decrease with work load.

However, CFFF is influenced by various factors (age, refractive error, activity, fasting, circadian rhythm etc.) and depends on the type of display, task, illumination, working distance. Therefore, CFFF cannot be treated as an absolute indicator of visual fatigue.

In addition, many of the above-listed parameters are also influenced by general fatigue and/or cognitive fatigue.

Therefore, not only is visual fatigue itself difficult to measure and assess objectively and precisely, but moreover, there is no known reliable objective metric or indicator of visual fatigue commonly agreed upon in the field.

Besides, it takes a long time to induce visual fatigue, e.g. more than one hour.

Therefore, in “real world” scenarios, like in a shop, it is not appropriate to build a tool to induce and measure visual fatigue.

Moreover, most known methods for calculating and predicting the visual fatigue level are about stereoscopic images, videos or virtual reality content viewing, but are limited to certain types of visual contents and are not suited for all kinds of visual tasks. Other known methods are solely based on eye image (eye open or eye closed). They do not take account of other input data.

Document CN 107468206 A discloses a stereoscopic content watching visual fatigue prediction method based on pupil diameter. Two models are described: one for predicting subjective visual fatigue level and one for predicting objective visual fatigue which is based solely on pupil diameter (i.e. it is predicting pupil diameter) after watching stereoscopic contents. Both models use a multiple-regression algorithm. Factors include the largest parallax, refreshing rate, environment brightness, watching angle and observer age. The output of the subjective model is the result of a visual fatigue questionnaire. The output of the objective model is pupil diameter.

Such solution is limited to stereoscopic content watching. Besides, the input data include parameters of the content and environment, as well as the age of the subject, but do not take into account objective parameters such as pupillary measurements. The pupil diameter is used as an output, but not as an input of the objective model. Moreover, objective and subjective predictions are in separate models, which makes the solution complex.

Furthermore, the prior art solutions merely focus on visual syndrome prediction and/or assessment. They do not make it possible to predict the change in visual fatigue, e.g. the increase thereof.

Therefore, there is an unfulfilled need for a tool that can quickly and easily predict the change, e.g. the increase, in the level of visual fatigue, for any kind of visual task, without itself causing visual fatigue.

### SUMMARY OF THE DISCLOSURE

An object of the disclosure is to overcome at least some of the above-mentioned limitations of the prior art and fulfill the above-mentioned need.

To that end, the disclosure provides a computer-implemented method for providing an automated prediction of a change of a state of fatigue of a subject carrying out a visual task involving any kind of visual content, comprising:
providing a plurality of input data, relating to said subject, to a fatigue state change predictive model, wherein the plurality of input data comprises at least one subjective measurement relating to the subject and/or at least one objective measurement relating to the subject and/or at least one other subject-related datum;
obtaining, by a processor implementing the model, a value representing a level of change of the state of fatigue of the subject.

Thus, different types of data, possibly including both subjective and objective measurements, are taken into account to predict the visual fatigue level change, be it for general digital near work or for any other visual task.

In addition, the method is easy and quick, e.g. it may take less than 5 minutes to obtain the value representing the fatigue state change level of the individual considered.

Not only does the method according to the disclosure save time, increase accuracy of the prediction and provide great simplicity of the process, but it also enables many kinds of preventative and alleviating functionalities.

Furthermore, the proposed model makes it possible to predict how easily a person can get fatigued, without having to do lengthy tasks to actually induce visual fatigue.

The model input data are measurements that may be done in a shop and take very little time, e.g. one or two minutes.

In addition, the output of the model, which represents the level of change of the state of fatigue of the considered individual, is generated immediately and can classify people into “fatiguers” of different levels. This makes it possible to make recommendations for the individual to buy an adapted anti-fatigue product.

Namely, to the same end, the disclosure also proposes a method for providing a subject with a personalized prescription of an anti-fatigue optical article, wherein the method comprises:
obtaining an automated prediction of a change of a state of fatigue of the subject carrying out a visual task involving any kind of visual content, by:
   providing a plurality of input data, relating to the subject, to a fatigue state change predictive model, wherein the plurality of input data comprises at least one subjective measurement relating to the subject and/or at least one objective measurement relating to the subject and/or at least one other subject-related datum;
   obtaining, by a processor implementing the model, a value representing a level of change of the state of fatigue of the subject;
providing the subject with an anti-fatigue optical article implementing a prescription adapted to the value representing the level of change of the state of fatigue of the subject.

To the same end, the disclosure also provides a device for providing an automated prediction of a change of a state of fatigue of a subject carrying out a visual task involving any kind of visual content, comprising:

a measuring unit, adapted to provide measurement input data comprising at least one subjective measurement relating to the subject and/or at least one objective measurement relating to the subject;
a processing unit, adapted to implement a fatigue state change predictive model using the measurement input data and/or at least one other subject-related datum to obtain a value representing a level of change of the state of fatigue of the subject;
a data inputting unit, adapted to obtain the measurement input data from the measuring unit and/or adapted to obtain the at least one other subject-related datum, the data inputting unit being adapted to input to the processing unit the measurement input data and/or the at least one other subject-related datum to cause the processing unit to implement the model;
a data outputting unit, adapted to output the value representing the level of change of the state of fatigue of the subject.

To the same end, the disclosure further provides an optometric machine comprising at least one device for providing an automated prediction of a change of a state of fatigue of a subject carrying out a visual task involving any kind of visual content, the at least one device comprising:
a measuring unit, adapted to provide measurement input data comprising at least one subjective measurement relating to the subject and/or at least one objective measurement relating to the subject;
a processing unit, adapted to implement a fatigue state change predictive model using the measurement input data and/or at least one other subject-related datum to obtain a value representing a level of change of the state of fatigue of the subject;
a data inputting unit, adapted to obtain the measurement input data from the measuring unit and/or adapted to obtain the at least one other subject-related datum, the data inputting unit being adapted to input to the processing unit the measurement input data and/or the at least one other subject-related datum to cause the processing unit to implement the model;
a data outputting unit, adapted to output the value representing the level of change of the state of fatigue of the subject.

To the same end, the disclosure further provides a computer program product comprising instructions that, when executed by a processor, cause the processor to obtain a value representing a level of change of a state of fatigue of a subject carrying out a visual task involving any kind of visual content, by using a fatigue state change predictive model, based on at least one subjective measurement relating to the subject and/or at least one objective measurement relating to the subject and/or at least one other subject-related datum provided as input data to the model.

To the same end, the disclosure further provides a mobile terminal, comprising a processor and a data storage unit, wherein the data storage unit contains instructions that, when executed by the processor, cause the processor to obtain a value representing a level of change of a state of fatigue of a subject carrying out a visual task involving any kind of visual content, by using a fatigue state change predictive model, based on at least one subjective measurement relating to the subject and/or at least one objective measurement relating to the subject and/or at least one other subject-related datum provided as input data to the model.

According to particular possible features, which may be combined or taken alone, of any of the methods, devices, machine, computer program product or mobile terminal succinctly described above:
the at least one subjective measurement may comprise data representing at least one answer by the subject to at least one question related to the vision of the subject,
the state of fatigue may be a state of visual fatigue,
the at least one objective measurement may comprise at least one pupillary and/or gaze measurement made on the subject,
the plurality of input data, respectively the measurement input data, may comprise at least one subjective and/or objective optometric measurement related to the subject,
the model may be stored in the cloud and the step of obtaining, by the processor implementing the model, the value representing the level of change of the state of fatigue of the subject may be carried out in the cloud,
the processor may be located in the cloud,
the predictive model may use machine learning,
the predictive model may use a classification algorithm and/or the value representing the level of change of the state of fatigue may be a discrete value,
the predictive model may use a regression algorithm and/or the value representing the level of change of the state of fatigue may be a continuous value,
the predictive model may be a machine learning-based predictive model.

As the method for providing a subject with a personalized prescription of an anti-fatigue optical article, as well as the device for providing an automated prediction of a change of a state of fatigue of a subject, the optometric machine, the computer program product and the mobile terminal succinctly described above have the same advantages as the method for providing an automated prediction of a change of fatigue of a subject, those advantages are not repeated here.

### BRIEF DESCRIPTION OF THE DRAWINGS

For a more complete understanding of the description provided herein and the advantages thereof, reference is now made to the brief descriptions below, taken in connection with the accompanying drawings and detailed description, wherein like reference numerals represent like parts.

FIG. 1 is a flow diagram showing steps of a method according to the disclosure for providing an automated prediction of a change of a state of fatigue of a subject carrying out a visual task, in a particular embodiment.

FIG. 2 is a schematic view of a device according to the disclosure for providing an automated prediction of a change of a state of fatigue of a subject carrying out a visual task, in a particular embodiment.

FIG. 3 is a schematic view of an optometric machine according to the disclosure, in a particular embodiment.

FIG. 4 is a schematic view of a mobile application implementing a method according to the disclosure, in a particular embodiment.

FIG. 5 is a simplified flow diagram of a method according to the disclosure for providing a subject with a personalized prescription of an anti-fatigue optical article, in a particular embodiment.

### DETAILED DESCRIPTION OF THE DISCLOSURE

In the description which follows, the drawing figures are not necessarily to scale and certain features may be shown in generalized or schematic form in the interest of clarity and conciseness or for informational purposes. In addition, although making and using various embodiments are discussed in detail below, it should be appreciated that as described herein are provided many inventive concepts that may embodied in a wide variety of contexts. Embodiments discussed herein are merely representative and do not limit the scope of the disclosure. It will also be obvious to one skilled in the art that all the technical features that are defined relative to a process can be transposed, individually or in combination, to a device and conversely, all the technical features relative to a device can be transposed, individually or in combination, to a process and the technical features of the different embodiments may be exchanged or combined with the features of other embodiments.

The terms “comprise” (and any grammatical variation thereof, such as “comprises” and “comprising”), “have” (and any grammatical variation thereof, such as “has” and “having”), “contain” (and any grammatical variation thereof, such as “contains” and “containing”), and “include” (and any grammatical variation thereof such as “includes” and “including”) are open-ended linking verbs. They are used to specify the presence of stated features, integers, steps or components or groups thereof, but do not preclude the presence or addition of one or more other features, integers, steps or components or groups thereof. As a result, a method, or a step in a method, that “comprises”, “has”, “contains”, or “includes” one or more steps or elements possesses those one or more steps or elements, but is not limited to possessing only those one or more steps or elements.

An optical article according to the disclosure comprises at least one ophthalmic lens or optical filter or optical glass or optical material suitable for human vision, e.g. at least one ophthalmic lens, or optical filter, or optical film or patch intended to be fixed on a substrate, or optical glass, or optical material intended for use in an ophthalmic instrument, for example for determining the visual acuity and/or the refraction of a subject, or any kind of safety device including a safety glass or safety wall intended to face an individual's eye, such as a protective device, for instance safety lenses or a mask or shield.

The optical article may be implemented as eyewear equipment having a frame that surrounds at least partially one or more ophthalmic lenses. By way of non-limiting example, the optical article may be a pair of glasses, sunglasses, safety goggles, sports goggles, a contact lens, an intraocular implant, an active lens with an amplitude modulation such as a polarized lens, or with a phase modulation such as an auto-focus lens, etc.

The at least one ophthalmic lens or optical glass or optical material suitable for human vision can provide an optical function to the user i.e. the wearer of the lens.

It can for instance be a corrective lens, namely, a power lens of the spherical, cylindrical and/or addition type for an ametropic user, for treating myopia, hypermetropia, astigmatism and/or presbyopia. The lens can have a constant power, so that it provides power as a single vision lens would do, or it can be a progressive lens having variable power.

In the present disclosure, the expression “visual fatigability” means the change of level of visual fatigue that may occur to a person, also referred to in the present disclosure as “a subject” or “an individual” or “a user” or “a wearer”, when that person is to use digital devices, or more generally, is to carry out any kind of visual task.

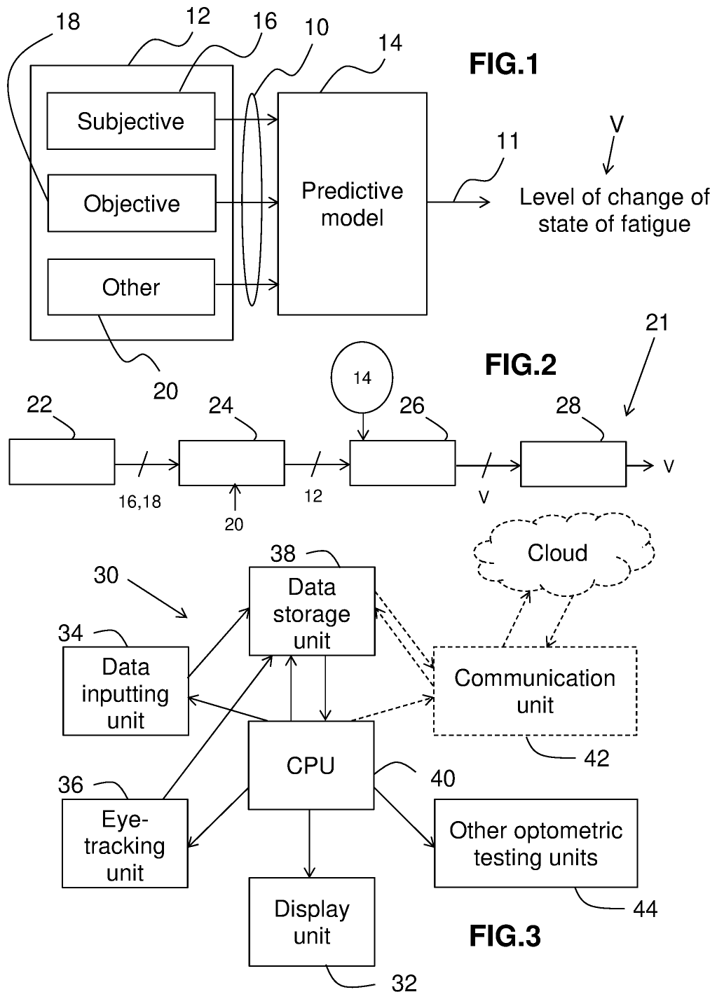

FIG. 1 shows the three major parts of both the method and, in functional terms, of the device according to the disclosure, in a particular embodiment, for providing an automated prediction of a change of a state of fatigue of a subject carrying out a visual task.

By way of non-limiting example, the state of fatigue may be a state of visual fatigue. Nevertheless, alternatively, as explained above, it may be another type of fatigue (cognitive, general, muscular, driver fatigue, etc.).

The visual task may involve any kind of visual content, e.g. it may be visual content displayed on a screen, or available on paper, or available otherwise in the environment viewed by the subject.

As shown in FIG. 1, the method comprises a step 10 of providing a plurality of input data 12 to a fatigue state change predictive model 14.

The input data 12 relate to the subject i.e. the person for which it is desired to provide the automated prediction of the change of the state of fatigue. The input data may comprise:
one or more subjective measurements 16 relating to the subject; and/or
one or more objective measurements 18 relating to the subject; and/or
one or more other data 20 relating to the subject.

The subjective measurements 16 differ from the objective measurements 18 by the method of measuring.

Therefore, a same type of parameter (such as near point of convergence, near point of accommodation, prism fusion range, etc.) can be subjective or objective depending on the way in which it is obtained.

The subjective measurements 16 are any results relying at least partially on a subject's response, which may be measured e.g. by a machine or an eye care professional.

Thus, the subjective measurements 16 may comprise data representing one or several answers made by the subject respectively to one or several questions related to the vision of that subject. By way of non-limiting example, the questions may regard the perception, by the subject, of his/her visual fatigue, current and general visual fatigue levels, etc. As a general visual fatigue questionnaire, the above-mentioned CISS may be used. Any appropriate current visual fatigue questionnaire may be used.

The objective measurements 18 are any results obtained e.g. by a machine or an eye care professional without utilizing any response made by any subject to any question asked by a machine or by an eye care professional.

Thus, the objective measurements 18 may comprise one or more pupillary measurements or any other ophthalmic measurements made on the subject by any suitable means that is known per se.

Pupillary data include for example pupil size, pupil micro-fluctuation, as well as changes thereof.

Ophthalmic measurements are common measurements that may be done by optometrists in shops and include, by way of non-limiting example, visual acuity, spherical equivalence, near point of convergence, near point of accommodation, prism fusion range and accommodative convergence/accommodation ratio.

The objective measurements 18 may comprise gaze measurements made on the subject by any suitable means that is known per se. For example, gaze measurements include gaze stability and fixation disparity, as well as blink-related measurements. They can be obtained in a few minutes and calculated with any eye-tracking device known per se.

The gaze measurements may be used, either in place of the pupillary measurements, or in addition to them.

The subjective and objective measurements can be taken simultaneously or not. When they are taken simultaneously, the method runs faster than when they are taken separately.

The other data 20 relating to the subject are subject-related data other than measurements, for example demographic data, gender, age, ethnic data, etc.

The plurality of input data 12 may comprise one or more subjective optometric measurements related to the subject, e.g. subjective refraction, near point of accommodation, the subjective way of measuring near point of convergence, prism fusion range, etc. More generally, any measurements involving, in the measuring process, asking the subject to report whether he/she can see something clearly or not, are considered as being subjective optometric measurements.

The plurality of input data 12 may comprise one or more objective optometric measurements related to the subject.

Any combination of any objective measurements is possible, as well as any combination of any subjective measurements, or any combination of any objective measurements with any subjective measurements.

Other types of data, such as testing time, viewing content, or postures may be added, in order to improve model performance and to extend the scenarios of using the method.

In addition, during the step 10 of providing the plurality of input data 12, the one or more subjective measurements 16 and the one or more objective measurements 18 may be made either simultaneously, or separately.

Using all the above-listed types of measurements and subject-related data will result in an even more precise and more powerful predictive model 14. However, it is not compulsory to use each and every one of those measurement and data types. Indeed, conversely, fewer types of data may be used, which may be more practical, but will result in lower performance. For example, for users not having access to pupillary measuring tools, with only subjective data and ophthalmic data it will still be possible to make predictions about visual fatigue level change. The reliability and performance of the predictive model 14 drops compared to the full model with all possible input data; but remains higher than the chance level.

As a non-limiting example, the input data involved in step 10 may be collected as follows. The above-mentioned questionnaires are run on a computer screen and eye-tracking measurements are taken at the same time. Such questionnaires take a short time, e.g. not more than 3 minutes.

From the eye-tracking data, pupillary parameters including blink rate, pupil size micro-fluctuation, gaze fixation disparity micro-fluctuation and gaze stability are calculated. In the next step of the analysis, a slope that represents the change in each of these parameters during the questionnaire answering is calculated. Together with the questionnaire results and the ophthalmic measurements which were taken initially, the predictive model 14 predicting the change in the result of the questionnaire of current subjective visual fatigue level is built.

Step 10 of providing the input data to the predictive model 14 is followed by a step 11 of obtaining, by a processor implementing the predictive model 14, a value V representing a level of change of the state of fatigue of the subject.

The processor may possibly, but not necessarily, use machine learning for implementing the predictive model 14.

The predictive model 14 may use a classification algorithm and/or the value V may be a discrete value ranging from a given lowest value to a given highest value.

As a variant, the predictive model 14 may use a regression algorithm and/or the value V may be a continuous value. For example, multiple regression may be used.

The value V makes it possible to classify any user into a predetermined number of categories, for example 3-5 categories, from low visual fatigability to high visual fatigability.

In the above-mentioned variant using a regression algorithm, the value V makes it possible to classify any user according to different levels of visual fatigability, e.g. by comparing the value V with values stored in a database.

In the above-mentioned embodiment using a classification algorithm, classification of users according to different levels of visual fatigability is obtained directly as a result of the algorithm. Thus, the method according to the disclosure may directly provide the category of the level of visual fatigability.

In embodiments where no machine learning is used, the predictive model 14 may use for example a simple linear regression.

Appropriate weights (in case of a regression machine learning-based algorithm) or other kinds of coefficients may advantageously be applied to each significant factor in order to have the best set of parameters for representing the largest part of the total variability of the data.

The predictive model 14 may also be based on a decision tree, a random forest, a support vector machine, neural networks, the well-known open source software library XGBoost, etc. As a non-limiting example, using the XGBoost classifier to try to classify visual fatigue change into high and low, the achieved overall accuracy, defined as the ratio of the number of correct predictions to the total number of input samples, was 73.91%.

In an embodiment, the method for providing an automated prediction of the fatigue state change may further comprise a step of storing the predictive model 14 in the cloud and a step of carrying out in the cloud the step 11 of obtaining the value representing the fatigue state change level of the considered subject.

The method according to the present disclosure can be as fast as a few minutes, for example around 3 minutes.

It predicts the change of level in visual fatigue without actually inducing or causing visual fatigue or its increment.

FIG. 2 shows a particular embodiment of a device 21 according to the disclosure for providing an automated prediction of a change of state of fatigue of a subject carrying out a visual task.

As in the above-described method, by way of non-limiting example, the state of fatigue considered by the device 21 may be a state of visual fatigue. Nevertheless, alternatively, as explained above, it may be another type of fatigue (cognitive, general, muscular, driver fatigue, etc.).

As in the above-described method, the visual task considered by the device 21 may involve any kind of visual content, e.g. it may be visual content displayed on a screen, or available on paper, or available otherwise in the environment viewed by the subject.

As shown in FIG. 2, the device 21 comprises a measuring unit 22, adapted to provide measurement input data.

The measurement input data comprise the subjective measurement(s) 16 relating to the subject and/or the objective measurement(s) 18 relating to the subject.

The device 21 further comprises a processing unit 26.

The processing unit 26 is adapted to implement the fatigue state change predictive model 14 using the measurement input data and/or the other datum/data 20 relating to the subject to obtain the value V representing the level of change of the state of fatigue of the subject. The processing unit 26 is also adapted to provide that value to a data outputting unit 28, which is described below.

As in the above-described method:
the subjective measurements 16 provided by the measuring unit 22 may comprise data representing one or several answers made by the subject respectively to one or several questions related to the vision of that subject;
the objective measurements 18 provided by the measuring unit 22 may comprise one or more pupillary measurements made on the subject by any suitable means that is known per se and that is included in the measuring unit 22;
the objective measurements 18 provided by the measuring unit 22 may comprise gaze measurements made on the subject by any suitable means that is known per se and that is included in the measuring unit 22;
the gaze measurements may be used, either in place of the pupillary measurements, or in addition to them;
the measurement input data 12 may comprise one or more subjective optometric measurements related to the subject.
the measurement input data 12 may comprise one or more objective optometric measurements related to the subject;
any combination of any objective measurements is possible, as well as any combination of any subjective measurements, or any combination of any objective measurements with any subjective measurements;
the processing unit 26 may possibly, but not necessarily, use machine learning for implementing the predictive model 14;
the predictive model 14 may use a classification algorithm and/or the value V may be a discrete value;
as a variant, the predictive model 14 may use a regression algorithm and/or the value V may be a continuous value.

The device 21 further comprises a data inputting unit 24.

The data inputting unit 24 is adapted to obtain the measurement input data from the measuring unit 22 and/or adapted to obtain the other datum/data 20 relating to the subject. The data inputting unit 24 is also adapted to input to the processing unit 26 the plurality of input data 12, that is to say, the measurement input data 16, 18 and/or the other datum/data 20 relating to the subject, to cause the processing unit 26 to implement the fatigue state change predictive model 14.

The device 21 further comprises the above-mentioned data outputting unit 28, which is adapted to output the value V representing the fatigue state change level of the subject.

In an embodiment, the processing unit 26 may be located in the cloud and the predictive model 14 is therefore implemented in the cloud.

In such an embodiment, the device 21 further comprises a communication unit (not shown in FIG. 2) adapted to communicate with the cloud. For example, the above-mentioned value V is received from the cloud via the communication unit.

The method, respectively the device according to the disclosure may be implemented, respectively integrated in a multifunction optometric machine which comprises at least one device as described above.

FIG. 3 shows an embodiment of such an optometric machine according to the disclosure.

In the particular embodiment shown in FIG. 3, the predictive model 14 is implemented in a multi-functional ophthalmic machine 30.

The machine 30 takes measurements of three types of input data, including questionnaire response, pupillary data with a built-in eye-tracker, as well as other ophthalmic parameters.

The machine 30 includes:
a display unit 32, which is for example a digital screen, to show the visual fatigue questionnaires and present testing results;
a data inputting unit 34 that records users' responses to the questionnaires and general information;
an eye-tracking unit 36 that measures pupillary and gaze parameters of the users;
a data storage unit 38;
a central processing unit (CPU) unit 40 that calculates visual fatigability with the predictive model 14 preloaded in the machine 30 and manages all the functionalities;
an optional data communication/transmission unit 42 that uploads data into the cloud and receives results about visual fatigability from the cloud;
a set 44 of units that collect other optometric parameters.

A non-limiting exemplary scenario of a practical use of the optometric machine 30 is as follows.

A customer comes into an optical shop. While waiting for the service for the next available optometrist or the salesperson, or if, in the conversation, the customer shows concerns, complaints or interests about visual fatigue, or if his or her profile fits “heavy digital user/worker”, the customer can be guided to do the test of visual fatigability on the optometric machine 30.

The test, which will usually last less than 3 minutes, collects data about responses to subjective visual fatigue questionnaires, as well as pupillary measurements with a built-in eye-tracker. The machine 30 is also used to do other ophthalmic measurements.

The CPU 40 then employs the predictive model 14 to do the calculation, either locally, or remotely in the cloud after the communication unit 42 uploads the data into the cloud and receives results of calculation back from the cloud. Almost instantaneously, the result of the visual fatigability test is shown on the display unit 32.

By way of non-limiting example, the result will determine the customer as having low, medium, high or very high fatigability. Corresponding to each level of fatigability, an anti-fatigue product will be recommended.

Thus, the visual fatigability test provides knowledge for both the customer and the practitioner about how easily the customer is to get visual fatigue when doing digital near work and whether he or she is in need of anti-fatigue products and other precaution measures to alleviate the condition.

As another non-limiting exemplary scenario (not shown in the drawings), a standing-alone visual fatigability toolbox implementing the predictive model 14 may be provided.

It may include the same units as described above for the machine 30, except the set of units 44.

In such a case, the practitioner needs to make ophthalmic measurements with separate machines and routines, before inputting the data with the data inputting unit 34 of the toolbox. In such a case, the practitioner may for example type in ophthalmic measurements into the system before or after the test.

The method according to the disclosure may be built into a mobile application installed in a mobile terminal.

In such embodiments:
the measurements may be taken by means of the mobile terminal;
data inputting may use the interface of the mobile terminal, e.g. keyboard or screen or other inputting or displaying means;
processing may be done by the central processing unit (CPU) of the mobile terminal carrying out calculation and prediction;
the displaying means of the mobile terminal may be used for showing results and recommendations to the user.

The predictive model 14 may be either pre-loaded into the mobile terminal, or stored in the cloud. Accordingly, the calculation may be done either locally in the mobile terminal, or remotely in the cloud.

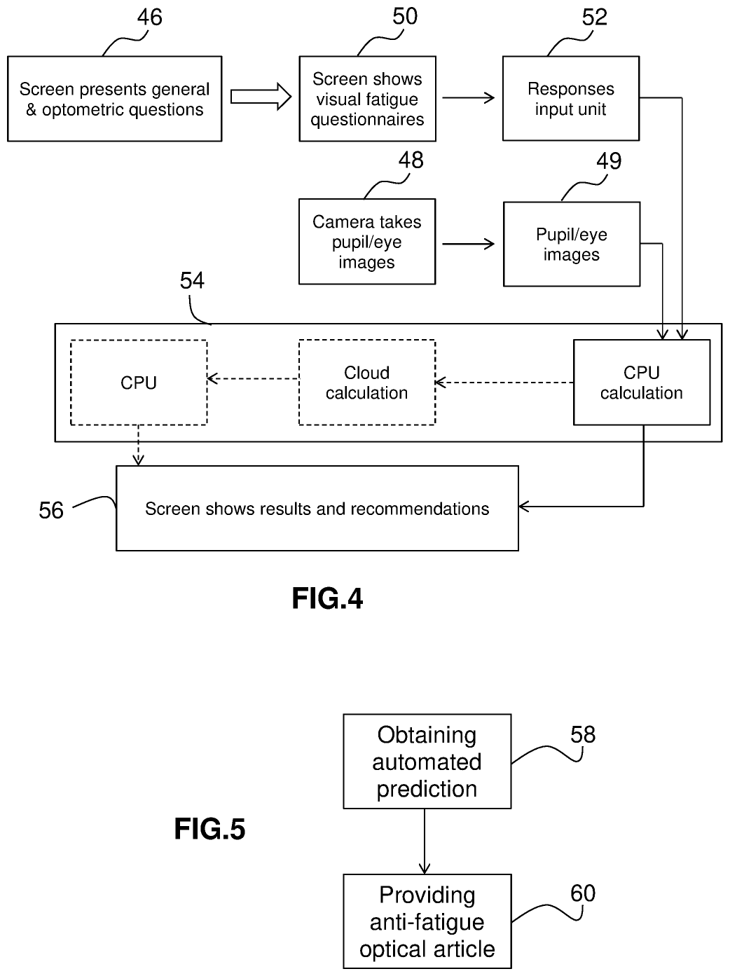

FIG. 4 shows still another non-limiting example, where the method according to the disclosure for providing an automated prediction of a change of a state of fatigue of a subject carrying out a visual task is implemented in a mobile application, which may be run on a digital device implementing the predictive model 14.

The functional units or flow of the mobile application include:
block 46: presenting visual fatigue questionnaires on the device screen;
block 48: at the same time recording pupils with the camera of the device and providing pupil/eye images 49;
block 50: presenting questions about general information and optometric measurements; this can be done at the beginning as well;
block 52: taking answers/input with the keyboard or touch screen of the device;
block 54: using the CPU of the device to do the calculation including pupillary parameters and visual fatigability prediction with the predictive model 14 preloaded in the mobile application, or, as a variant, uploading data into the cloud and receiving results about visual fatigability from the cloud with the communication unit of the mobile device;
block 56: presenting the prediction V on the screen of the device.

The practitioners may use the mobile application with their own mobile device and input general information as well as optometric data into the mobile application.

As still another example of usage of the method according to the disclosure, a Web site directed to visual fatigability may be made available to users, employing for example a simplified version of the predictive model 14, where users can easily answer visual fatigue questionnaires and input general information and optometric data.

This is a downgraded version with respect to the devices or the mobile application in the earlier examples, for practitioners who do not necessarily have access to optometric machines or devices with an eye-tracking function, or cameras on their mobile devices, but who may use those mobile devices for pupillary measurements.

The Web site gets the data input by the user, performs calculation of a simplified version of the visual fatigability level and shows results regarding the visual fatigability level and recommendations similarly to the previous examples.

As still another example, a Web site directed to visual fatigability may be made available to users, employing the simplified visual fatigability model, where users can answer visual fatigue questionnaires and input general information and basic optometric data like visual acuity, spherical equivalence, etc.

This is a downgraded version with respect to the devices or the mobile application in the earlier examples, for ordinary consumers or the general public, who do not have access to optometric machines or devices with an eye-tracking function, or cameras on their mobile devices, which may nevertheless be used for pupillary measurements.

In this last example, the Web site gets the data input by the user, performs calculation with an even more simplified version of the visual fatigability model and shows results regarding the visual fatigability level and recommendations similarly to the previous examples.

Thus, the method according to the disclosure can be used in consumers' everyday life to monitor their general visual fatigability level and to send alerts about a recent increase in visual fatigability, and potentially recommend anti-fatigue practice and products.

Adding the monitoring function of the pupillary measurement, it can also alert about an increase of visual fatigue before it actually kicks in, therefore reminding users to take breaks.

Furthermore, it can help ophthalmologists in their practice with identifying patients with high visual fatigability, in order to prescribe practice and products to improve the condition.

Besides, it can be used among children and teenagers who are in the time window of myopia progression, to alert about visual fatigue and excessive digital device usage/near work, in order to help with myopia control.

Moreover, an adjusted version of the model can be used on drivers, to predict a fatigue level increase before it actually kicks in, to alert the drivers, therefore reducing driving in fatigue and improving road safety. Such an adjusted version may for example include a method for monitoring driver's pupillary and gaze behaviour constantly, or at least periodically and frequently.

Similarly, the method according to the disclosure can be used in factories or any other work environment, in particular where safety requirements are high and linked to fatigue level.

In addition, the method according to the disclosure can be used as a tool to filter subjects for scientific or consumer studies, to select or exclude certain subjects that exhibit high levels of visual fatigability, to improve the quality of study of populations, according to specific study purposes.

As shown in FIG. 5, the method according to the disclosure for providing a subject with a personalized prescription of an anti-fatigue optical article comprises a step 58 of obtaining an automated prediction of a change of a state of fatigue of the subject carrying out a visual task involving any kind of visual content, by carrying out above-described steps of the method according to the disclosure for providing an automated prediction of a change of the state of fatigue of the subject and a step 60 of providing the subject with an anti-fatigue optical article implementing a prescription adapted to the value V.

For example, adapting the prescription to the value V may comprise linking the value V to the level of near addition of anti-fatigue lenses, e.g. the higher the value V, the higher the level of near addition.

By way of non-limiting example, the anti-fatigue article may be a pair of glasses and/or contact lenses and/or eye drops and/or behavioural trainings and/or orthoptics sessions and/or may take any other appropriate form.

The computer program product according to the disclosure comprises instructions that, when executed by a processor that may operate as described above, cause the processor to obtain the value V, by using the predictive model 14, based on at least one subjective measurement relating to the subject and/or at least one objective measurement relating to the subject and/or at least one other subject-related datum provided as input data to the predictive model 14.

The mobile terminal according to the disclosure comprises a processor that may operate as described above and a data storage unit. The data storage unit contains instructions that, when executed by the processor, cause the processor to obtain the value V, by using the predictive model 14, based on at least one subjective measurement relating to the subject and/or at least one objective measurement relating to the subject and/or at least one other subject-related datum provided as input data to the predictive model 14.

Although representative methods and devices have been described in detail herein, those skilled in the art will recognize that various substitutions and modifications may be made without departing from the scope of what is described and defined by the appended claims.

1. A computer-implemented method for providing an automated prediction of a change of a state of fatigue of a subject carrying out a visual task involving any kind of visual content, comprising:
providing a plurality of input data, relating to said subject, to a fatigue state change predictive model, wherein said plurality of input data comprises at least one subjective measurement relating to said subject and/or at least one objective measurement relating to said subject and/or at least one other subject-related datum;
obtaining, by a processor implementing said model, a value representing a level of change of said state of fatigue of said subject.

2. A method according to claim 1, wherein said at least one subjective measurement comprises data representing at least one answer by said subject to at least one question related to the vision of said subject.

3. A method according to claim 1, wherein said state of fatigue is a state of visual fatigue.

4. A method according to claim 1, wherein said at least one objective measurement comprises at least one pupillary and/or gaze measurement made on said subject.

5. A method according to claim 1, wherein said plurality of input data comprises at least one subjective and/or objective optometric measurement related to said subject.

6. A method according to claim 1, wherein it further comprises storing said model in the cloud and carrying out said obtaining in the cloud.

7. A method for providing a subject with a personalized prescription of an anti-fatigue optical article, wherein said method comprises:
obtaining an automated prediction of a change of a state of fatigue of said subject carrying out a visual task involving any kind of visual content, by:
providing a plurality of input data, relating to said subject, to a fatigue state change predictive model, wherein said plurality of input data comprises at least one subjective measurement relating to said subject and/or at least one objective measurement relating to said subject and/or at least one other subject-related datum;
obtaining, by a processor implementing said model, a value representing a level of change of said state of fatigue of said subject;
providing said subject with an anti-fatigue optical article implementing a prescription adapted to said value representing said level of change of said state of fatigue of said subject.

8. A device for providing an automated prediction of a change of a state of fatigue of a subject carrying out a visual task involving any kind of visual content, comprising:
a measuring unit, adapted to provide measurement input data comprising at least one subjective measurement relating to said subject and/or at least one objective measurement relating to said subject;
a processing unit, adapted to implement a fatigue state change predictive model using said measurement input data and/or at least one other subject-related datum to obtain a value representing a level of change of said state of fatigue of said subject;
a data inputting unit, adapted to obtain said measurement input data from said measuring unit and/or adapted to obtain said at least one other subject-related datum, said data inputting unit being adapted to input to said processing unit said measurement input data and/or said at least one other subject-related datum to cause said processing unit to implement said model;
a data outputting unit, adapted to output said value representing said level of change of said state of fatigue of said subject.

9. A device according to claim 8, wherein said at least one subjective measurement comprises data representing at least one answer by said subject to at least one question related to the vision of said subject.

10. A device according to claim 8 or 9, wherein said state of fatigue is a state of visual fatigue.

11. A device according to claim 9, wherein said at least one objective measurement comprises at least one pupillary and/or gaze measurement made on said subject.

12. A device according to claim 8, wherein said measurement input data comprise at least one subjective and/or objective optometric measurement related to said subject.

13. An optometric machine, wherein said machine comprises at least one device for providing an automated prediction of a change of a state of fatigue of a subject carrying out a visual task involving any kind of visual content, said at least one device comprising:
a measuring unit, adapted to provide measurement input data comprising at least one subjective measurement relating to said subject and/or at least one objective measurement relating to said subject;
a processing unit, adapted to implement a fatigue state change predictive model using said measurement input data and/or at least one other subject-related datum to obtain a value representing a level of change of said state of fatigue of said subject,
a data inputting unit, adapted to obtain said measurement input data from said measuring unit and/or adapted to obtain said at least one other subject-related datum, said data inputting unit being adapted to input to said processing unit said measurement input data and/or said at least one other subject-related datum to cause said processing unit to implement said model;
a data outputting unit, adapted to output said value representing said level of change of said state of fatigue of said subject.

14. A computer program product comprising instructions that, when executed by a processor, cause said processor to obtain a value representing a level of change of a state of fatigue of a subject carrying out a visual task involving any kind of visual content, by using a fatigue state change predictive model, based on at least one subjective measurement relating to said subject and/or at least one objective measurement relating to said subject and/or at least one other subject-related datum provided as input data to said model.

15. A mobile terminal, comprising a processor and a data storage unit, wherein said data storage unit contains instructions that, when executed by said processor, cause said processor to obtain a value representing a level of change of a state of fatigue of a subject carrying out a visual task involving any kind of visual content, by using a fatigue state change predictive model, based on at least one subjective measurement relating to said subject and/or at least one objective measurement relating to said subject and/or at least one other subject-related datum provided as input data to said model.
